(19) United States
(12) Patent Application Publication
(10) Pub. No.: US 2021/0102913 A1
(43) Pub. Date: Apr. 8, 2021

(54) COMPOSITIONS AND METHODS OF DETECTING 17 BETA-ESTRADIOL

(71) Applicant: CASE WESTERN RESERVE UNIVERSITY, Cleveland, OH (US)

(72) Inventors: Chung-Chiun Liu, Cleveland Heights, OH (US); Yifan Dai, Cleveland Heights, OH (US); Laurie Dudik, South Euclid, OH (US)

(21) Appl. No.: 16/499,757

(22) PCT Filed: Mar. 29, 2018
(86) PCT No.: PCT/US2018/025275

§ 371 Date: Sep. 30, 2019

Related U.S. Application Data
(60) Provisional application No. 62/478,138, filed on Mar. 29, 2017.

(51) Int. Cl. G01N 27/327; G01N 33/487

(52) U.S. Cl. CPC G01N 27/3277; G01N 33/48707

(57) ABSTRACT

A sensor for the detection of 17β-estradiol in a sample includes a substrate, a working electrode and counter electrode formed on a surface of the substrate, and an anti-estrogen receptor functionalized or chemically functionalized to a surface of an exposed portion of the working electrode.

### RELATED APPLICATION

This application claims priority from U.S. Provisional Application No. 62/478,138, filed Mar. 29, 2017, the subject matter of which is incorporated herein by reference in its entirety.

### BACKGROUND

Estrogen is a steroid hormone which is directly responsible for the development and regulation of the female reproductive system. Furthermore, estrogen is considered to be carcinogenic and has a tumor promotion effect. Its level related to the risk of breast cancer is evident. In terms of human health, the estrogen level in women is also related to lung cancer, uterine (endometrial) and ovarian cancers, even though the exact mechanism of the cancer development is not entirely understood. It is also well recognized that, psychologically, the level of the estrogen in women can affect weight gain, depression, fatigue, mood swings, trouble sleeping and others. Consequently, an estrogen or estradiol test or physician-prescribed estrogen therapy may be helpful in addressing the impact of estrogen levels in women.

Estrogen contamination in the environment due to the large quantity of natural estrogen from human urine disturbs the endocrine system in the ecosystem, and it is well recognized. Pollution of the environment and food supply caused by estrogenic chemicals are well acknowledged. It is well documented that estrogen pollution causes the death and deformation of birds, fishes, animals as well as human beings. Specifically, the Water Framework Directive (WFD) of the European Union listed 17β-estradiol as a priority pollutant of estrogens. Therefore, for both biomedical and environmental health reasons, the detection of estrogen is of scientific and health importance.

There are instrumental analysis techniques of measuring estrogen, including high-performance liquid chromatography (HPLC), gas chromatography/mass spectroscopy (GC/MS) and others. These analyses are very sensitive and accurate, but are also very complicated to perform, requiring expensive instruments and well-trained operators. Consequently, a simpler and less expensive measurement technology of estrogen will be of scientific and commercial importance. Biosensors are one of the potential technologies which can minimize the shortcomings of the current detection technologies mentioned above, providing a simpler and sensitive detection method of estrogen.

### SUMMARY

Embodiments described herein relate to a system and/or method for detecting, indentifying, quantifying, and/or determining the amount or level of 17β-estradiol in a sample, and particularly relates to system, which includes an electrochemical biosensor, for detecting, identifying, quantifying, and/or determining the amount or level of 17β-estradiol in a sample, such as water or other fluids (e.g., urine). The system and method described herein can provide a single use, disposable, and cost-effective means for simple assessment of 17β-estradiol in water and biological samples obtained by non-invasive or minimally invasive means.

The system and methods described herein includes an electrochemical biosensor, a redox solution, and a measuring device. The electrochemical biosensor can produce a signal that is related to the presence or quantity of the 17β-estradiol being detected in a sample. In some embodiments, the system can be used to detect and/or quantify 17β-estradiol that is present in tap or drinking water or a biological fluid, such as urine.

In some embodiments, the electrochemical biosensor includes a substrate, a working electrode formed on a surface of the substrate and a counter electrode formed on the surface of the substrate. A dielectric layer covers a portion of the working electrode and counter electrode and defines an aperture exposing other portions of the working electrode and counter electrode. Ann anti-estrogen receptor is functionalized or chemically functionalized to a surface of the exposed portion of the working electrode. The anti-estrogen receptor selectively binds to 17β-estradiol in a sample, and the 17β-estradiol once bound is detectable by measuring the current flow between the working electrode and counter electrode.

The redox solution is applied to the working electrode for determining the quantity of 17β-estradiol in the sample bound to the anti-estrogen receptor. The measuring device applies voltage potentials to the working electrode and counter electrode and measures the current flow between the working electrode and counter electrode to determine the level of the 17β-estradiol in a sample, such as a drinking water, tap water, or urine.

In some embodiments, the working electrode and the counter electrode include metalized films. The metalized films used to form the working electrode and the counter electrode can independently comprise gold, platinum, palladium, silver, carbon, alloys thereof, and composites thereof. The metalized films can be provided on the surface of the substrate by sputtering or coating the films on the surface and then laser ablating the films to form the working electrode and counter electrode.

In other embodiments, the sensor can include a reference electrode on the surface of the substrate. The dielectric can cover a portion of the reference electrode.

In other embodiments, the anti-estrogen receptor can be chemically functionalized to the surface of the working electrode coated with a 3-mercaptopropionic acid (MPA) monolayer. The anti-estrogen receptor can include an α-estrogen antibody.

### BRIEF DESCRIPTION OF THE DRAWINGS

FIG. 1 is a schematic illustration of a biosensor in accordance with an aspect of the application.

FIG. 2 illustrates a plot showing DPV measurements of DMSO and PBS solution indicating that DMSO as a solvent for 17β-estradiol will not contribute to any current output in DPV measurement as compared to that in PBS. DPV, differential pulse voltammetry; DMSO, Dimethyl sulfoxide; PBS, Phosphate buffer saline.

FIGS. 3(A-B) illustrate plots showing (A) a DPV measurement of 17β-estradiol over the concentration range of 2.25-2250 pg/mL in 0.1 M PBS solution; (B) Calibration curve of the DPV outputs and 17β-estradiol concentration in 0.1 M PBS solution. Anti-estrogen receptor concentration is 45 μg/mL.

FIGS. 4(A-B) illustrate plots showing (A) DPV measurements of 17β-estradiol antigen in the tap water samples; (B) The calibration curve of the 17β-estradiol antigen detection in tap water samples based on the DPV measurement from FIG. 3a with n=3.

FIGS. 5(A-B) illustrate plots showing (A) DPV measurement of 17β-estradiol antigen over the concentration range of 2.25 to 2250 pg/mL in simulated urine; (B) Calibration curve of the DPV outputs and 17β-estradiol concentration in simulated urine. Anti-estrogen receptor concentration is 45 μg/mL.

FIG. 6 illustrates a plot showing DPV measurements of estradiol 17 at the concentration of 225 pg/mL and 2250 pg/mL in the presence and absence of equal quantity of testosterone.

### DETAILED DESCRIPTION

Unless specifically addressed herein, all terms used have the same meaning as would be understood by those of skilled in the art of the subject matter of the application. The following definitions will provide clarity with respect to the terms used in the specification and claims.

As used herein, the term “monitoring” refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication.

As used herein, the term “quantitative data” or “quantitative level” or “quantitative amount” refers to data, levels, or amounts associated with any dataset components (e.g., markers, clinical indicia) that can be assigned a numerical value.

As used herein, the term “subject” refers to a human or another mammal. Typically, the terms “subject” and “patient” are used herein interchangeably in reference to a human individual.

As used herein, the term “bodily sample” refers to a sample that may be obtained from a subject (e.g., a human) or from components (e.g., tissues) of a subject. The sample may be of any biological tissue or fluid with, which analytes described herein may be assayed. Frequently, the sample will be a “clinical sample”, i.e., a sample derived from a patient. Such samples include, but are not limited to, bodily fluids, e.g., saliva, breath, urine, blood, plasma, or sera; and archival samples with known diagnosis, treatment and/or outcome history. The term biological sample also encompasses any material derived by processing the bodily sample. Processing of the bodily sample may involve one or more of, filtration, distillation, extraction, concentration, inactivation of interfering components, addition of reagents, and the like.

As used herein, the terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal.

Embodiments described herein relate to a system and/or method for detecting, indentifying, quantifying, and/or determining a quantitative amount or level of 17β-estradiol in a sample, and particularly relates to system, which includes an electrochemical biosensor, for detecting, identifying, quantifying, and/or determining the quantitative amount or level of 17β-estradiol in a sample, such as water or other fluids (e.g., urine). The system and method described herein can provide a single use, disposable, and cost-effective means for simple assessment of 17β-estradiol in water and biological samples obtained by non-invasive or minimally invasive means.

The system and methods described herein include an electrochemical biosensor, a redox solution, and a measuring device. The electrochemical biosensor can produce a signal that is related to the presence or quantity of the 17β-estradiol being detected in a sample. In some embodiments, the system can be used to detect and/or quantify 17β-estradiol that is present in tap or drinking water or a biological fluid, such as urine.

In some embodiments, the electrochemical biosensor includes a substrate, a working electrode formed on a surface of the substrate and a counter electrode formed on the surface of the substrate. A dielectric layer covers a portion of the working electrode and counter electrode and defines an aperture exposing other portions of the working electrode and counter electrode. An anti-estrogen receptor is functionalized or chemically functionalized to a surface of the exposed portion of the working electrode. The anti-estrogen receptor selectively binds to anti-17β-estradiol antibody in a sample, and the 17β-estradiol once bound is detectable by measuring the current flow between the working electrode and counter electrode.

The redox solution is applied to the working electrode for determining the quantity of 17β-estradiol in the sample bound to the anti-estrogen receptor. The measuring device applies voltage potentials to the working electrode and counter electrode and measures the current flow between the working electrode and counter electrode to determine the level of the 17β-estradiol in a sample, such as a drinking water, tap water, or urine.

The bio-recognition mechanism of this sensor is based on the influence of the redox coupling reaction of the redox solution, such as a potassium ferrocyanide/potassium ferricyanide (K₃Fe(CN)₆/K₄Fe(CN)₆) solution, by the 17β-estradiol antigen and its α-receptor (ER-α; α-estrogen antibody). In the detection of 17β-estradiol, the estrogen receptor α (ER-α; α-estrogen antibody) is used to provide a lock-and-key bio-recognition mechanism. This α-estrogen interacts with 17β-estradiol affecting the electron charge transfer and can influence a redox coupling reaction in the redox solution applied to the working electrode. The level of 17β-estradiol bound to the anti-estrogen receptor can be determined by measuring current flow between the working and counter electrode to which the sample and redox solution has been applied and comparing the measured current to control value, which can be based on a measured current between the working electrode and counter electrode that is free of bound 17β-estradiol.

Differential pulse voltammetry (DPV) can employed as the transduction mechanism of this biosensor to determine the level of bound 17β-estradiol. DPV applies a linear sweep voltammetry with a series of regular voltage pulses superimposed on the linear potential sweep. The current can then measured immediately before each potential change. Thus, the effect of the charging current could be minimized, achieving a higher sensitivity.

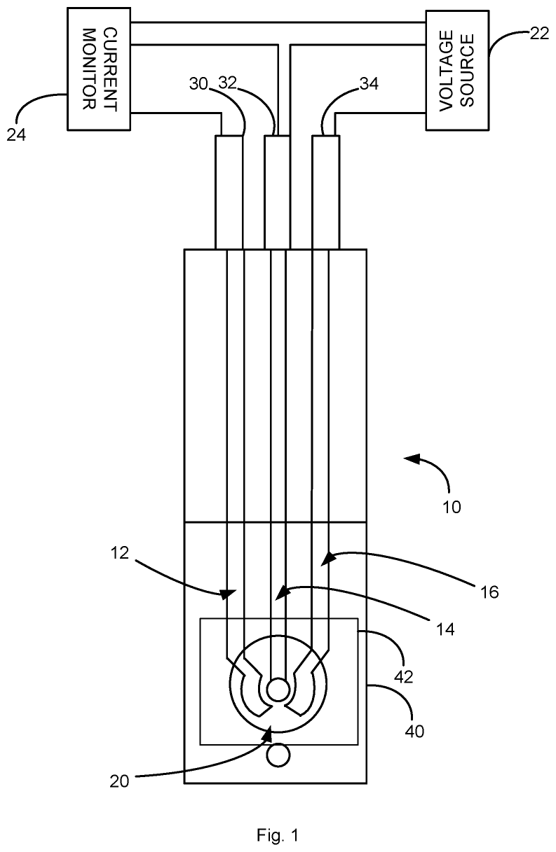

FIG. 1 illustrates a biosensor 10 of the system in accordance with an embodiment of the application. The sensor 10 is a three-electrode sensor including a counter electrode 12, a working electrode 14, and a reference electrode 16 that are formed on a surface of a substrate. A dielectric layer 40 covers a portion of the working electrode 12, counter electrode 14 and reference electrode 16. The dielectric layer 40 includes an aperture 20 that defines a detection region of the working electrode 12, counter electrode 14, and reference electrode 16, which is exposed to samples containing 17β-estradiol to be detected. An anti-estrogen receptor for 17β-estradiol can be functionalized or chemically functionalized to the working electrode. The anti-estrogen receptor can bind selectively to 17β-estradiol in the biological sample.

The system further includes a measuring device that includes a voltage source 22 for applying a voltage potential to the working electrode, counter electrode, and/or reference electrode and a current monitor 24 for measuring the current flow between the working electrode and counter electrode.

The interaction of the anti-estrogen receptor and 17β-estradiol in the presence of a redox solution can be detected using electrochemical analytical techniques, such as cyclic voltammetry (CV), differential pulse voltammetry (DPV), to determine the presence of the analyte in the sample. The working electrode 14 is poised at an appropriate electrochemical potential such that the current that flows through the electrode changes when the anti-estrogen receptor binds to 17β-estradiol in the sample in the presence of the redox solution. The function of the counter electrode 12 is to complete the circuit, allowing charge to flow through the sensor 10.

The working electrode 14 and the counter electrode 12 are preferably formed of the same material, although this is not a requirement. Examples of materials that can be used for the working electrode 14 and counter electrode 12 include, but are not limited to, gold, platinum, palladium, silver, carbon, alloys thereof, and composites thereof.

The anti-estrogen receptor, which is functionalized or chemically functionalized to the working electrode, can be an antibody that binds selectively to 17β-estradiol. An antibody that binds selectively to 17β-estradiol can be a monoclonal or polyclonal α-estrogen antibody that binds selectively or specifically to 17β-estradiol. An α-estrogen antibody having binding affinities in the picomolar to micromolar range are suitable. Such interaction can be reversible or irreversible.

The term “functionalized” or “chemically functionalized,” as used herein, means addition of functional groups onto the surface of a material by chemical reaction(s). As will be readily appreciated by a person skilled in the art, functionalization can be employed for surface modification of materials in order to achieve desired surface properties, such as biocompatibility, wettability, and so on. Similarly, the term “biofunctionalization,” “biofunctionalized,” or the like, as used herein, means modification of the surface of a material so that it has desired biological function, which will be readily appreciated by a person of skill in the related art, such as bioengineering.

The anti-estrogen receptor may be functionalized to the working electrode covalently or non-covalently. Covalent attachment of an anti-estrogen receptor to the working electrode may be direct or indirect (e.g., through a linker). Anti-estrogen receptors may be immobilized on the working electrode using a linker. The linker can be a linker that can be used to link a variety of entities.

In some embodiments, the linker may be a homo-bifunctional linker or a hetero-bifunctional linker, depending upon the nature of the molecules to be conjugated. Homo-bifunctional linkers have two identical reactive groups. Hetero-bifunctional linkers have two different reactive groups. Various types of commercially available linkers are reactive with one or more of the following groups: primary amines, secondary amines, sulphydryls, carboxyls, carbonyls and carbohydrates. Examples of amine-specific linkers are N-hydroxysuccinimide (NHS), bis(sulfosuccinimidyl) suberate, bis[2-(succinimidooxycarbonyloxy)ethyl]sulfone, disuccinimidyl suberate, disuccinimidyl tartarate, N-succinimidyl S-acetylthioacetate, dimethyl adipimate 2HCl, dimethyl pimelimidate 2HCl, dimethyl suberimidate HCl, ethylene glycolbis-[succinimidyl-[succinate]], dithiolbis(succinimidyl propionate), and 3,3′-dithiobis(sulfosuccinimidylpropionate). Linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Linkers preferentially reactive with carbohydrates include azidobenzoyl hydrazine. Linkers preferentially reactive with carboxyl groups include 4-[p-azidosalicylamido]butylamine.

Heterobifunctional linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional linkers that react with carboxyl and amine groups include 1-ethyl-3-[3-dimethylaminopropyl]-carbodiimide hydrochloride. Heterobifunctional linkers that react with carbohydrates and sulfhydryls include 4-[N-maleimidomethyl]-cyclohexane-1-carboxylhydrazide HCl, 4-(4-N-maleimidophenyl)-butyric acid hydrazide 2HCl, and 3-[2-pyridyldithio]propionyl hydrazide.

Alternatively, α-estrogen antibodies may be non-covalently coated onto the working electrode. Non-covalent deposition of the α-estrogen antibody to the working electrode may involve the use of a polymer matrix. The polymer may be naturally occurring or non-naturally occurring and may be of any type including but not limited to nucleic acid (e.g., DNA, RNA, PNA, LNA, and the like, or mimics, derivatives, or combinations thereof), amino acids (e.g., peptides, proteins (native or denatured), and the like, or mimics, derivatives, or combinations thereof, lipids, polysaccharides, and functionalized block copolymers. The α-estrogen antibody may be adsorbed onto and/or entrapped within the polymer matrix.

Alternatively, the α-estrogen antibody may be covalently conjugated or crosslinked to the polymer (e.g., it may be “grafted” onto a functionalized polymer).

An example of a suitable peptide polymer is poly-lysine (e.g., poly-L-lysine). Examples of other polymers include block copolymers that comprise polyethylene glycol (PEG), polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(lactide-glycolide), copolyoxalates, polycaprolactones, polyesteramides, polyorthoesters, polyhydroxybutyric acid, polyanhydrides, poly(styrene-b-isobutylene-b-styrene) (SIBS) block copolymer, ethylene vinyl acetate, poly(meth)acrylic acid, polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, albumin and other hydrophilic proteins, and other prolamines and hydrophobic proteins, copolymers and mixtures thereof, and chemical derivatives thereof including substitutions and/or additions of chemical groups, for example, alkyl, alkylene, hydroxylations, oxidations, and other modifications routinely made by those skilled in the art.

In one particular embodiment, the working electrode can comprise a gold working electrode that is coated with a self-assembled monolayer (SAM) of 3-mercaptopropionic acid (MPA). The MPA molecule includes a thiol functional group at one end with an affinity for gold and a carboxylic group at the other end, which can covalently bond to proteins through a peptide bond after activation. The SAM of MPT can be activated by reaction with N-(3-dimethylaminopropyl)-N′-ethylcarbodiimide hydrochloride (EDC) and N-hydroxysuccinimide (NHS), which can further react with amine groups of proteins and antibodies.

In some embodiments, the α-estrogen antibody can include monoclonal and polyclonal antibodies, immunologically active fragments (e.g., Fab or (Fab)2 fragments), antibody heavy chains, humanized antibodies, antibody light chains, and chimeric antibodies. α-estrogen antibody, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czemik et al., NeuroNeuroprotocols, 1995, 6: 56-61; H. Zhang et al., J. Biol. Chern. 2002, 277: 39379-39387; S. L. Morrison et al., Proc. Natl. Acad. Sci., 1984, 81: 6851-6855; M. S. Neuberger et al., Nature, 1984, 312: 604-608; S. Takeda et al., Nature, 1985, 314: 452-454). Antibodies to be used in the biosensor can be purified by methods well known in the art (see, for example, S. A. Minden, “Monoclonal Antibody Purification”, 1996, IBC Biomedical Library Series: Southbridge, Mass.). For example, α-estrogen antibodies can be affinity purified by passage over a column to which a protein marker or fragment thereof is bound. The bound antibodies can then be eluted from the column using a buffer with a high salt concentration.

Instead of being prepared, α-estrogen antibodies to be used in the methods described herein may be obtained from scientific or commercial sources.

In order to minimize any non-specific binding on the working electrode surface and blocking any open surface area of the working electrode at least one blocking agent can be applied to the surface of the working electrode once the α-estrogen antibody has been functionalized or chemically functionalized to the working electrode. The blocking agent can enhance the reproducibility and sensitivity of the biosensor by minimizing non-specific interactions on the working electrode. In some embodiments, the blocking agent can include dithiothreitol or casein. The blocking agent can be applied to the surface of the working at an amount effective to minimize non-specific binding of proteins or other molecules on the surface of the working electrode.

The redox solution is applied to the working electrode for determining the quantity of 17β-estradiol in the sample bound to the anti-estrogen receptor. The redox coupling solution can include a redox mediator, such as potassium ferrocyanide/potassium ferricyanide (K₃Fe(CN)₆/K₄Fe(CN)₆), that is provided at equimolar concentration in a PBS solution.

The voltage source 22 can apply a voltage potential to the working electrode 14 and reference and/or counter electrode 16, 12, depending on the design of the sensor 10. The current between the working electrode 14 and counter electrode 16 can be measured with the measuring device or meter 24. Such current is dependent on interaction of 17β-estradiol in the sample with the anti-estrogen receptor on the working electrode.

The amount or level of current measured is proportional to the level or amount of 17β-estradiol in the sample. In some embodiments, where the sample is a tap water or drinking water sample, once the current level generated by the sample and redox solution tested with the sensor is determined, the level can be compared to a predetermined value or control value to provide information for monitoring the presence or absence of estrogen in the water sample.

In other embodiments, where the sample is a bodily sample obtained from a subject, once the current level generated by the reaction solution tested with the sensor is determined, the level can be compared to a predetermined value or control value to provide information for diagnosing or monitoring of the condition, pathology, or disorder in a subject that is associated with presence or absence of estrogen.

The current level generated by sample obtained from the subject can be compared to a current level of a sample previously obtained from the subject, such as prior to administration of a therapeutic. Accordingly, the methods described herein can be used to measure the efficacy of a therapeutic regimen for the treatment of a condition, pathology, or disorder associated with the level of the estrogen in a subject by comparing the current level obtained before and after a therapeutic regimen. Additionally, the methods described herein can be used to measure the progression of a condition, pathology, or disorder associated with the presence or absence of the estrogen in a subject by comparing the current level in a bodily sample obtained over a given time period, such as days, weeks, months, or years.

The current level generated by a sample obtained from a subject may also be compared to a predetermined value or control value to provide information for determining the severity or aggressiveness of a condition, pathology, or disorder associated with estrogen levels in the subject. A predetermined value or control value can be based upon the current level in comparable samples obtained from a healthy or normal subject or the general population or from a select population of control subjects.

The predetermined value can take a variety of forms. The predetermined value can be a single cut-off value, such as a median or mean. The predetermined value can be established based upon comparative groups such as where the current level in one defined group is double the current level in another defined group. The predetermined value can be a range, for example, where the general subject population is divided equally (or unequally) into groups, or into quadrants, the lowest quadrant being subjects with the lowest current level, the highest quadrant being individuals with the highest current level. In an exemplary embodiment, two cutoff values are selected to minimize the rate of false positive and negative results.

The biosensor illustrated in FIG. 1 can be fabricated on a substrate 100 formed from polyester or other electrically non-conductive material, such as other polymeric materials, alumina (Al₂O₃), ceramic based materials, glass or a semi-conductive substrate, such as silicon, silicon oxide and other covered substrates. Multiple sensor devices can thus be formed on a common substrate. As will be appreciated, variations in the geometry and size of the electrodes are contemplated.

The biosensor can be made using a thin film, thick film, and/or ink-jet printing technique, especially for the deposition of multiple electrodes on a substrate. The thin film process can include physical or chemical vapor deposition. Electrochemical sensors and thick film techniques for their fabrication are discussed in U.S. Pat. No. 4,571,292 to C. C. Liu et al., U.S. Pat. No. 4,655,880 to C. C. Liu, and co-pending application U.S. Ser. No. 09/466,865, which are incorporated by reference in their entirety.

In some embodiments, the working electrode, counter electrode, and reference electrode may be formed using laser ablation, a process which can produce elements with features that are less than one-thousandth of an inch. Laser ablation enables the precise definition of the working electrode, counter electrode, and reference electrode as well as electrical connecting leads and other features, which is required to reduce coefficient of variation and provide accurate measurements. Metalized films, such as Au, Pd, and Pt or any metal having similar electrochemical properties, that can be sputtered or coated on plastic substrates, such as PET or polycarbonate, or other dielectric material, can be irradiated using laser ablation to provide these features.

In one example, a gold film with a thickness of about 300 A to about 2000 A can be deposited by a sputtering technique resulting in very uniform layer that can be laser ablated to form the working and counter electrodes. The counter electrode can use other materials. However, for the simplicity of fabrication, using identical material for both working and counter electrodes will simplify the fabrication process providing the feasibility of producing both electrodes in a single processing step. An Ag/AgCl reference electrode, the insulation layer, and the electrical connecting parts can then be printed using thick-film screen printing techniques.

The working electrode surface can then be cross-linked or biotinylated chemically in order to allow the attachment of an anti-estrogen receptor. The crosslinking step can be accomplished by generating thiol bonds. This can be chemically accomplished using, for example, a self-assembled monolayer (SAM) of 3-mercaptopropionic acid (MPA). The MPA molecule includes a thiol functional group at one end with an affinity for gold and a carboxylic group at the other end, which can covalently bond to proteins through peptide bond after activation. The SAM of MPT can be activated for binding to a protein, such as an antibody, by reaction with N-(3-dimethylaminopropyl)-N′-ethylcarbodiimide hydrochloride (EDC) and N-hydroxysuccinimide (NHS) that can further react with amine groups of proteins and antibodies. Similar chemical methods can be used to produce semi-stable amine-ester groups to enhance the cross linking between the antibodies and the thiol groups. Other cross-linking agent, such as 3,3′-dithiobis[sulfosuccinimidylpropionate] (DTSSP), can also be used in this process.

Biotinylation is rapid, specific and is normally unperturb to the natural function of the molecule due to the relatively small size of biotin. Streptavidin and similar chemicals such as avidin can be immobilized on the working electrode surface for a biosensor for the detection of an interaction of anti-estrogen receptor and 17β-estradiol.

Following addition of an anti-estrogen receptor to the working electrode, the working electrode surface can be blocked using a blocking agent to minimize any non-specific molecule (e.g., protein) bonding on the electrode surface. This step will enhance the reproducibility and sensitivity of the biosensor. In some embodiments, DTT (Dithiothreitol), casein, and/or other blocking agents can be used to cover the open surface area of the working electrode and minimize any non-specific protein coverage.

In other embodiments, a plurality of biosensors can be provided on a surface of a substrate to provide a biosensor array. The biosensor array can be configured to detect 17β-estradiol concentration changes in a host of chemical and/or biological processes occurring in proximity to the array. The biosensor array can include a plurality biosensors arranged in a plurality of rows and a plurality of columns. Each biosensor can use a working electrode, a counter electrode, and a dielectric layer covering a portion of the working electrode and counter electrode and defining an aperture exposing other portions of the working electrode and counter electrode. Anti-estrogen receptors for 17β-estradiol can be functionalized or chemically functionalized to the working electrode. The anti-estrogen receptors can be the same or different for each biosensor of the array and can bind selectively to 17β-estradiol. The biosensors of the array can be configured to provide at least one output signal representing the presence and/or concentration of 17β-estradiol proximate to a surface of the array. For each column of the plurality of columns or for each row of the plurality of rows, the array further comprises column or row circuitry configured to provide voltage potentials to respective biosensors in the column or row. Each biosensor in the row or column can potentially detect a different analyte and/or biased to detect different analytes.

### Example 1

In this Example, we show the development of a simple, cost-effective detection method of 17β-estradiol. Specifically, a cost-effective, single-use, in vitro or in situ 17β-estradiol detection biosensor is developed for this practical application. This 17β-estradiol biosensor is portable and simple to operate, and suitable for both health care and environmental applications.

Biosensor uses for the measurement of 17β-estradiol have been exploited by different groups of researchers. These reported approaches have their own merits and limitations. In some cases, the sensitivity of the detection was limited. In other cases, the quantitation of nano-gold particles used for each single electrode element of the biosensor was difficult, making the practical applications of the estrogen biosensor impossible and expensive. The transduction mechanism of this example was differential pulse voltammetry (DPV), which required 30 s for a complete measurement; while others used electrochemical impedance spectroscopy (EIS) or AC impedance measurement would require 600 s or longer. Thus, our DPV measurement was much more time-efficient. Furthermore, our thin gold film-based electrode was prepared by sputtering physical vapor deposition, which was accomplished on an atomic level deposition, providing uniform and reproducible electrode surface and higher sensor sensitivity. In order to minimize the shortcomings in detecting 17β-estradiol, a cost-effective, single-use, disposable biosensor for practical applications is undertaken in this research.

In this Example, the bio-recognition mechanism of this biosensor was based on the influence of the redox coupling reaction, K₃Fe(CN)₆/K₄Fe(CN)₆ by the 17β-estradiol antigen and its α-receptor (ER-α; α-estrogen antibody). Antibody and antigen interaction was a “lock-and-key” one-to-one combination providing the specificity of the biosensor. In the detection of 17β-estradiol, the estrogen receptor α (ER-α; α-estrogen antibody) is used to provide this lock-and-key bio-recognition mechanism. This α-estrogen interacts with 17β-estradiol affecting the electron charge transfer and can influence a redox coupling reaction in the test medium. Consequently, the level of 17β-estradiol can be assessed.

The fabrication of the biosensor used in this example employed sputtering—a physical vapor deposition (PVD) technique—to formulate the thin-film gold working and counter electrode elements of the biosensor; it was deposited at an atomic level resulting in the very uniform and reproducible electrode elements. This fabrication step could also be accomplished on a roll-to-roll manufacturing process. This biosensor had a three-electrode configuration, and the reference electrode was a thick-film printed Ag/AgCl electrode. Laser ablation technique was used to define the structure and size of the biosensor elements.

Differential pulse voltammetry (DPV) of electrochemical analytical technique was employed as the transduction mechanism of this biosensor. DPV applied a linear sweep voltammetry with a series of regular voltage pulses superimposed on the linear potential sweep. The current was then measured immediately before each potential change. Thus, the effect of the charging current could be minimized, achieving a higher sensitivity. Furthermore, the K₃Fe(CN)₆/K₄Fe(CN)₆ redox coupling reaction was used, demonstrating the effect of 17β-estradiol and α-estrogen antibody interaction in the test medium. It was based on the unique design and fabrication of the biosensor and the application of DPV measurement that this cost-effective, single-use, disposable in vitro and in situ biosensor for estrogen, specifically 17β-estradiol, was successfully developed. Phosphate buffer saline (PBS), normal tap water (from the Cleveland regional water district) and simulated urine were used as the test media. These tests demonstrated that this biosensor could be used for both human care and environmental applications. 17β-estradiol in the concentration range of 2.25-2250 pg/mL was used in this study, covering a wide range of 17β-estradiol concentrations.

### Materials and Methods

### Apparatus and Reagents

Phosphate Buffer Solution (PBS) 1.0 M (pH 7.4) (Cat. #P3619), 3-Mercaptopropionic acid (MPA) (Cat. #5801), N-(3-dimethylaminopropyl)-N′-ethylcarbodiimide hydrochloride (EDC) (Cat. #E1769), and N-hydroxysuccinimide (NHS) (Cat. #130672) were purchased from Sigma-Aldrich (St. Louis, Mo., USA). 17β-estradiol (Cat. #E8875) was also obtained from Sigma-Aldrich (St. Louis, Mo., USA) and anti-estrogen receptor, α-antibody [E-115] of estrogen (Cat. #ab32063) was purchased from ABCAM (Cambridge, Mass., USA). Potassium hydroxide pellets (Cat. #P1767), concentrated H₂SO₄ 95.0 to 98.0 w/w % (Cat. #A300) and concentrated HNO₃ 70% w/w % (Cat. #A200) were received from Fisher Scientific (Pittsburgh, Pa., USA). Dimethyl sulfoxide (DMSO) (Cat. #BP231-1) was also obtained from Fisher Scientific (Pittsburgh, Pa., USA). Simulated urine, normal (Cat. #695955) was purchased from the Carolina Biological Supply Co. (Burlington, N.C., USA). For the interference study, testosterone C-111N (Cat. #T1500) from Sigma-Aldrich (St. Louis, Mo., USA) was obtained. Testosterone was a controlled substance and required special permission to obtain the chemical. All the chemicals were used without further purification. A CHI 660C (CH Instrument, Inc., Austin, Tex., USA) Electrochemical Workstation was used for DPV and EIS investigations. Similar Model CHI 660 A-E Electrochemical Workstations could also be used. All the experiments were conducted at room temperature. X-ray Photoelectron Spectroscopy (XPS) was performed by a PHI Versaprobe 5000 Scanning X-Ray Photoelectron Spectrometer.

### Biosensor Fabrication

This estrogen biosensor was based on a platform that was designed and manufactured. This biosensor used a three-electrode configuration. Both working and counter electrodes were thin gold film of 50 nm in thickness. The thin gold film was deposited using roll-to-roll sputtering technique. This roll-to-roll process was an established industrial process in which each sensor was estimated to cost less than US$2 to manufacture. Hence, the process was very cost-effective and the gold electrode elements were very uniform and reproducible, which were very practical and unique for single-use, in vitro or in situ applications. The overall dimensions of an individual biosensor were 33.0×8.0 mm². The working electrode area was 1.54 mm², accommodating 10-15 μL of liquid test sample. The employment of known micro-fabrication processes, such as sputtering physical vapor deposition, laser ablation and thick-film printing techniques, resulted in producing high-reproducible and low-cost, single-use disposable biosensors. As mentioned, a more detailed explanation of the electrode fabrication process can be found elsewhere.

### Chemical Modification of the Biosensor

### Pretreatment of Gold Electrode (AuE)

As reported previously, a pretreatment procedure was applied to the gold electrode, prior to the MPA-SAM deposition. This three-step pretreatment procedure resulted in a significant decrease in electrode charge transfer resistance, enhancing the reproducibility of the biosensor. A row of five or seven biosensors was immersed in a 2 M KOH solution for 15 min. After rinsing with copious amounts of deionized water, the biosensors were placed in a 0.05 M H₂SO₄ solution (95.0 to 98.0 w/w %) for another 10 min. DI water was then used to rinse the biosensor prototypes. The biosensors were then placed in a 0.05 M HNO₃ solution (70 w/w %) for another 10 min. The biosensors were rinsed one more time with DI water and dried gently in a steam of nitrogen. The purpose of this pretreatment of the biosensor was to ensure the reproducibility of the biosensor, and the electrochemical impedance spectroscopy (EIS) study confirmed that this chemical pretreatment step was very effective. K₃Fe(CN)₆/K₄Fe(CN)₆ with 5 mM in each component was prepared in 0.1 M KCl for the EIS study. Concentrations of acids and base solutions used in this pretreatment procedure were optimized to be effective while maintaining the integrity of the thin gold film working and counter electrodes and the Ag/AgCl reference electrode, as well as the overall structure of the biosensor. The effectiveness of the pretreatment procedure was assessed using EIS and the results were excellent.

### Chemical Immobilization Steps on the Gold Electrode (AuE)

In this step, a thiol group was applied in order to provide a linkage between the anti-estrogen receptor and the gold electrode surface. Self-assembled monolayers of 3-Mercaptopropionic acid (MPA) were used for this purpose. MPA molecule consisted of a thiol functional group at one end, which provided an excellent affinity to gold, and a carboxylic group at another end, which was suitable for bonding covalently to proteins through peptide bond after an activation procedure. Thiol modification of gold electrode surface for protein immobilization was a well-acknowledged technique. Typically, 4-8 biosensors were prepared in this immobilization step as a batch for this study. The biosensors were immersed in 50 mM solution of MPA in ethanol for 24 h in the dark, rinsed with DI water and dried in a steam of N₂. The carboxylic groups on the other end of the MPA-modified AuEs were then functionalized by incubating in 0.1 M PBS (pH=7.4) containing 0.25 M EDC and 0.05 M NHS for 5 h. Activated AuEs were then rinsed by 0.1 M PBS and dried by N₂ flow; 20 μL of 45 μg/mL anti-estrogen receptor was casted on the sensing area of each AuE and left to dry overnight at 4° C. Antibody immobilized biosensors were rinsed with 0.1 M PBS and immersed in 0.5 mM bovine serum albumin (BSA) in 0.1 M PBS solution for 2 h, preventing non-specific bonding. The biosensors were then rinsed with 0.1 M PBS again, dried under a steam of N₂ and stored at 4° C.

### Characterization of the Biosensor

Prior to actual application, the characterization of the prepared biosensor was necessary to ensure that the biosensors were properly modified as designed. This investigation involved (1) the electrochemical analysis of bare, MPA-SAM-modified and antibody-attached biosensors; and (2) the degree of completeness in covering the biosensor in the chemical immobilization process.

In the electrochemical analysis of the biosensor at different stages of the modification, a solution of K₃Fe(CN)₆ and K₄Fe(CN)₆, with 5 mM in each component, was prepared in 0.1 M PBS and used as the redox coupled probe for DPV and EIS tests. In DPV measurement, it was anticipated that the bare biosensor would have the highest current output. Subsequently, the MPA-SAM- and antibody-modified biosensors would have lower current output indicating that the modification steps were successful. This observation was identical to that obtained in other biomarker detection of the platform biosensor technology. EIS tests were performed in the Frequency range of 10⁻² to 10⁴ Hz with 5 mV voltage amplitude. Randles equivalent circuit models were used to fit the Nyquist plots of EIS using EC-lab standard software.

X-ray photoelectron spectroscopy was used in the assessment of the degree of completeness in covering biosensors through the chemical process. Similar to our study of this platform biosensor, XPS high-resolution spectra of C(1s) and S(2p) obtained for MPA-SAM-modified AuE at the take-off angles of 10°, 50° and 90° were examined. The experimental results confirmed that there were fewer numbers of carboxylic groups near the surface. This observation confirmed the upward orientation of MPA-SAM carboxylic groups in this MPA-SAM arrangement as identical to the data given in previous study of this platform biosensor.

### Results

### Preparation of Different Concentrations of 17β-Estradiol Testing Solution

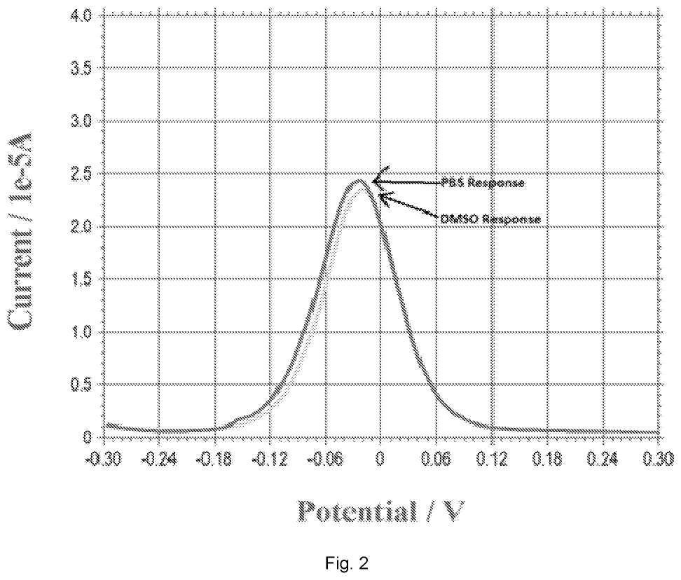

17β-estradiol had a limited solubility in PBS, distilled water and other aqueous solutions. However, it can be dissolved completely in dimethyl sulfoxide (DMSO). Consequently, 17β-estradiol was first dissolved in DMSO in order to prepare different concentrations of 17β-estradiol for testing. Thus, any potential effect of DMSO in the electrochemical measurement must first be assessed. Experimentally, differential pulse voltammetry (DPV) of our biosensor in pure DMSO and in 0.1 M PBS solution were carried out and the results were compared. FIG. 2 shows the DPV measurement in DMSO and PBS solution. The nearly identical current outputs in the DPV measurements, as shown in FIG. 2, suggest that DMSO did not contribute to any electrochemical effect as compared to PBS in DPV measurement using this biosensor. Similarly, 17β-estradiol dissolved in DMSO would not contribute to any electrochemical current in tap water and simulated urine test solutions.

### 17β-Estradiol Detection in 0.1 M PBS

The detection of 17β-estradiol was based on the effect on the redox reaction, K₃Fe(CN)₆/K₄Fe(CN)₆ affecting by the interaction between 17β-estradiol and its α anti-estrogen receptor. The anti-estrogen receptor used in this study was α-antibody of estrogen. Differential pulse voltammetry (DPV) was used in this study. The reaction between 17β-estradiol and α-antibody of estrogen was irreversible. Thus, the DPV measurement of this interaction measured only the Faradic current, which was a diffusional control reaction influenced by the concentration of the 17β-estradiol. Furthermore, DPV waves were affected by parameters, including the electrode reaction rate constant, transfer coefficient, waveform parameters. Consequently, the minor potential shift of the DPV waveform was due to these factors.

The MPA and EDC+NHS-modified biosensor was then attached with α-antibody of estrogen. The concentration of α-antibody of estrogen used was 45 μg/mL. The concentration of the 17β-estradiol antigen used in this study was in the range of 2.25-2250 pg/mL. Preparation of the 17β-estradiol in the PBS required a carefully developed procedure; 0.02 g of 17β-estradiol antigen was placed in 1 mL of DMSO, 10 μL of this 17β-estradiol-DMSO mixture was then added to 30 mL of PBS, And 10 μL of this solution was then added to 30 mL of PBS, resulting in a 2250 pg/mL of 17β-estradiol in PBS. One mL of this 2250 pg/mL solution was then added into 9 mL PBS, resulting in a 225 pg/mL 17β-estradiol in PBS. Concentrations of 17β-estradiol in PBS of 22.5 pg/mL and 2.25 pg/mL were prepared in a similar manner, sequentially. The biosensor was prepared with the α-receptor antibody, then 20 μL of the 17β-estradiol antigen in PBS was placed on top of the biosensor. The biosensor was then incubated at room temperature for three hours and then rinsed with 0.1 M PBS and dried with N₂ gas. A redox solution, K₃Fe(CN)₆/K₄Fe(CN)₆ was prepared using 5 mM equally of K₃Fe(CN)₆ and K₄Fe(CN)₆ in 0.1 M PBS solution; 20 μL of this redox solution was then added on top of the biosensor, and DPV measurement was then made.

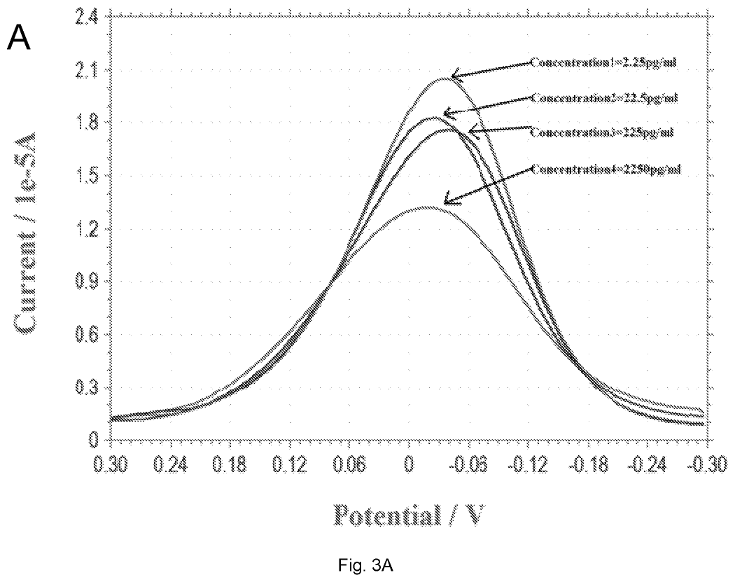

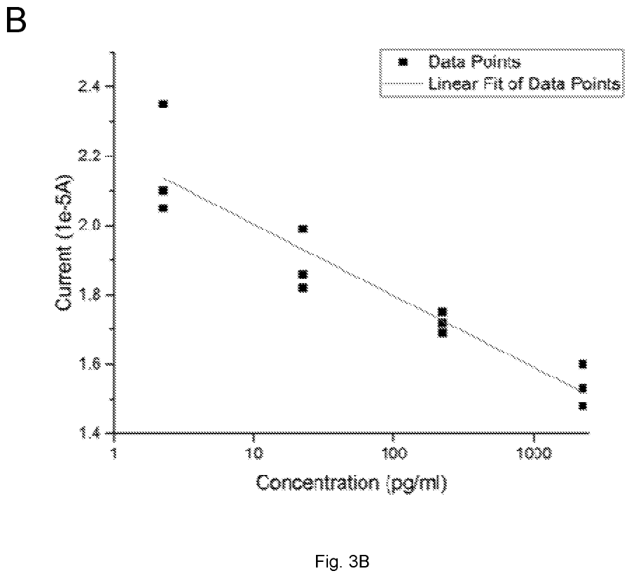

FIG. 3A shows the DPV measurements of 17β-estradiol antigens in 0.1 M PBS solution and FIG. 3B shows the calibration curve based on the DPV measurements in FIG. 3A. All the measurements from FIG. 3 were conducted by the single-use disposable biosensor.

17β-Estradiol Detection in Tap Water from Cleveland, Ohio Regional Water District

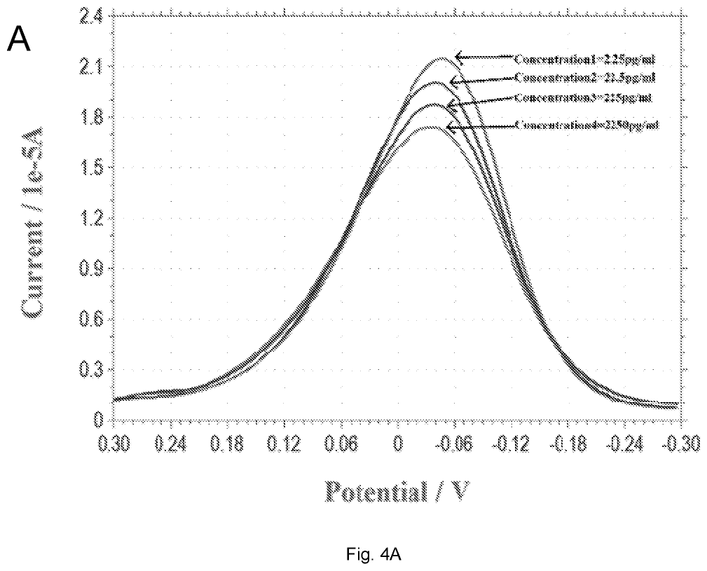

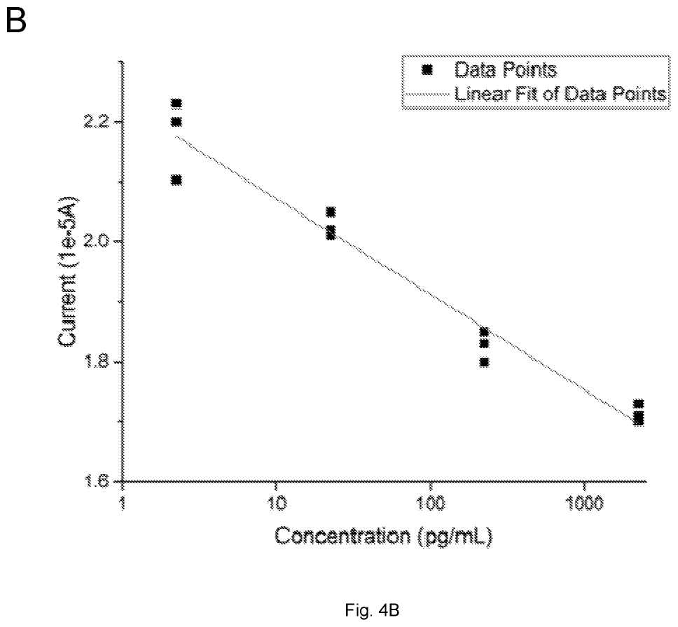

Estrogen pollution is an environmental concern, and the goal of this study includes the development of a simple in situ biosensor for 17β-estradiol detection in regular water systems. Therefore, the regular tap water from the Cleveland regional water district was used as a test medium. 17β-estradiol antigen was used to spike the tap water test sample providing the range of the 17β-estradiol antigen for detection in a typical tap water sample. The range of the concentration of 17β-estradiol antigen in the tap water sample was 2.25-2250 pg/mL, which was prepared in the same manner as in the PBS solution. DPV measurements of the 17β-estradiol antigen were similar to the measurements of 17β-estradiol antigen in PBS. FIG. 4A shows the DPV measurement of the current outputs of the biosensor covering the 17β-estradiol antigen concentration range of 2.25-2250 pg/mL in tap water from the Cleveland regional water district. FIG. 4B is the calibration curve based on the DPV measurements from FIG. 3a with n=3.

### 17β-Estradiol Detection in Simulated Urine Test Sample

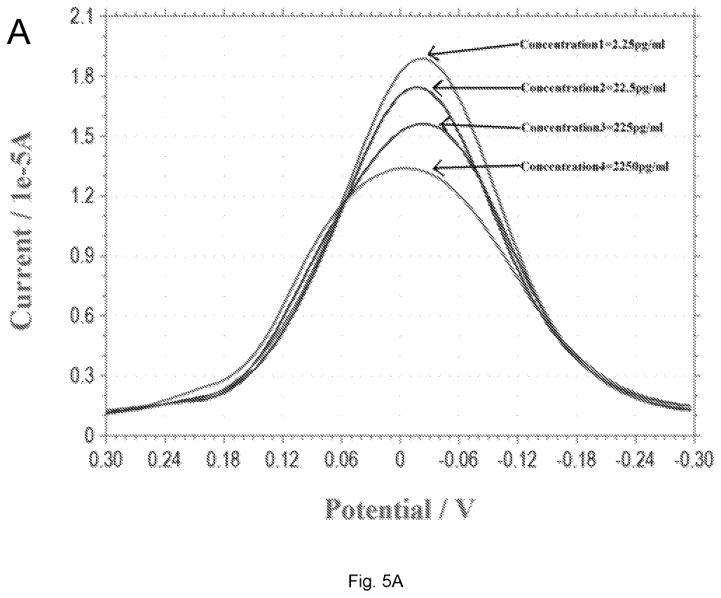

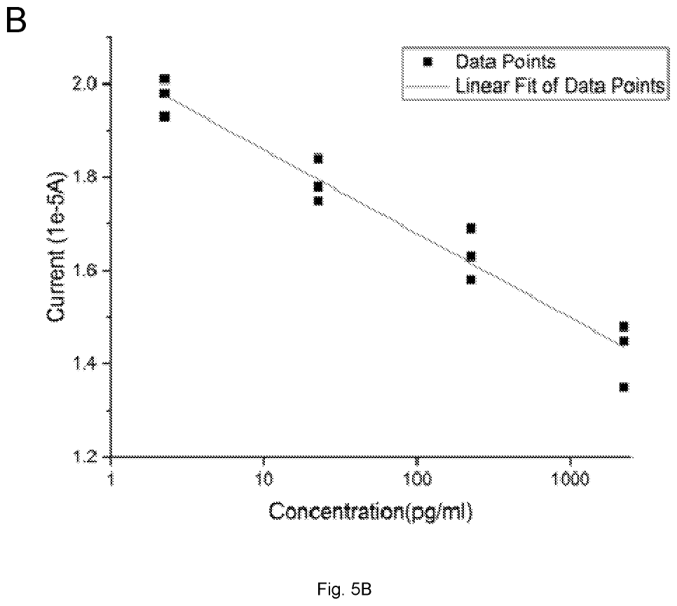

Estrogen is directly related to the health of humans, particularly women. While the health implication of estrogen to woman is beyond the scope of this study, the development of a single-use in vitro biosensor for 17β-estradiol antigen detection applicable to health care was one of the main focuses of this study. Specifically, this biosensor should be simple to use and would not require expensive instruments or skillful operators. In this aspect, simulated urine, normal (Cat. #695955) was purchased from the Carolina Biological Supply Co. (Burlington, N.C., USA) and used. Urine sample is a non-invasive clinical procedure and it is very practical for in vitro testing. FIG. 5A shows the 17β-estradiol antigen measurements in the simulated urine samples using DPV measurements. The 17β-estradiol antigen concentration range was 2.25-2250 pg/mL. FIG. 5B is the calibration curve based on the DPV measurement from FIG. 4a with n=3.

Interference Study of this 17β-Estradiol Biosensor

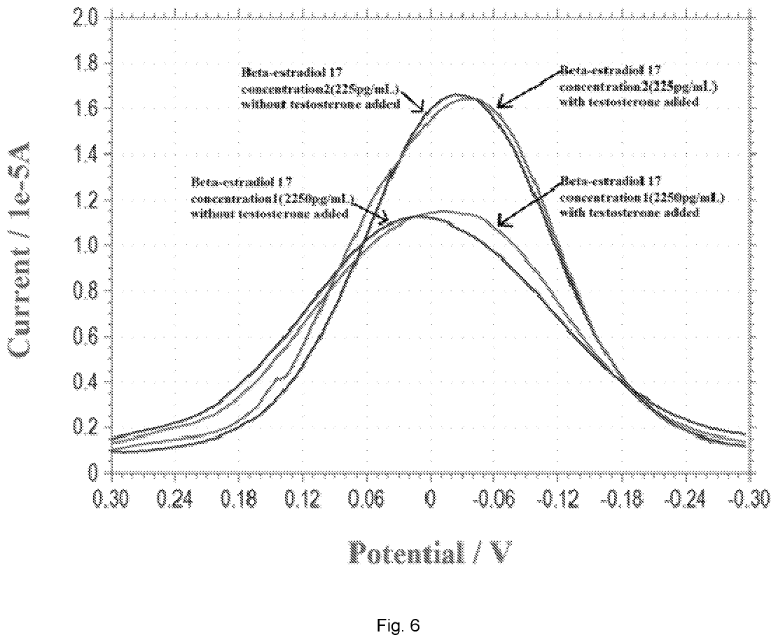

The selectivity and specificity of a biosensor is important in any meaningful development of a biosensor. This suggests that the biosensor should not be subject to interference by other hormones or biomarkers while in use. In this example, we chose testosterone as a potential interference in the detection of 17β-estradiol. The justification of selecting testosterone was based on the similar chemical structure between 17β-estradiol and testosterone, C₁₈H₂₄O₂ and C₁₉H₂₈O₂, respectively. Also, the molecular weights between 17β-estradiol, 272.388 g/mol and testosterone, 288.431 g/mol were close and were useful in this interference study. In this phase of the study, four different 17β-estradiol antigen concentrations were used, namely, 2.25 pg/mL, 22.5 pg/mL, 225 pg/mL and 2250 pg/mL. At each 17β-estradiol antigen concentration, an equal quantity of testosterone was then added into the test medium. PBS was used as the test medium. The current outputs of the DPV measurement of the biosensor in the presence and absence of the testosterone were nearly the same, indicating that testosterone will not interfere with this 17β-estradiol biosensor, and suggesting that the selectivity of this biosensor based on the bio-recognition mechanism was very good and unique. FIG. 6 shows the selected results of this interference study. Only the interference studies at 17β-estradiol concentrations of 225 pg/mL and 2250 pg/mL are shown in FIG. 6. The current outputs of the DPV measurements in the presence and the absence of testosterone are identical. The biosensor was used only once and was disposable. The performance as shown in FIG. 6 not only demonstrates the good selectivity (non-interference) of this biosensor, but also the repeatability of this 17β-estradiol biosensor.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention the following is claimed:

1. A detection system for detecting 17β-estradiol levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-estrogen receptor functionalized or chemically functionalized to a surface of the exposed portion of the working electrode, the anti-estrogen receptor selectively binding to anti-17β-estradiol antibody in a sample and the 17β-estradiol once bound being detectable by measuring the current flow between the working electrode and counter electrode,
a redox solution that is applied to the working electrode for determining the quantity of 17β-estradiol in the sample bound to the anti-estrogen receptor, and
a measuring device for applying voltage potentials to the working electrode and counter electrode and measuring the current flow between the working electrode and counter electrode.

2. The system of claim 1, wherein the working electrode and the counter electrode comprise metalized films.

3. The system of claim 2, wherein the working electrode and counter electrode independently comprise gold, platinum, palladium, silver, carbon, alloys thereof, and composites thereof.

4. The system of claim 2, wherein the metalized films are provided on the surface of the substrate by sputtering or coating the films on the surface and wherein the working electrode and the counter electrode are formed using laser ablation to define the dimensions of the working electrode and the counter electrode.

5. The system of claim 1, wherein the redox solution comprises potassium ferrocyanide/potassium ferricyanide solution.

6. The system of claim 1, further comprising a reference electrode on the surface of the substrate, the dielectric covering a portion of the reference electrode.

7. The system of claim 1, the anti-estrogen receptor being chemically functionalized to the surface of the working electrode coated with a 3-mercaptopropionic acid (MPA) monolayer.

8. The system of claim 1, wherein the anti-estrogen receptor comprises an α-estrogen antibody.

9. The system of claim 1, wherein the sample comprises urine or tap water.

10. A detection system for detecting 17β-estradiol levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-estrogen receptor functionalized or chemically functionalized to a surface of the exposed portion of the working electrode, the anti-estrogen receptor selectively binding to anti-17β-estradiol antibody in a sample and the 17β-estradiol once bound being detectable by measuring the current flow between the working electrode and counter electrode,
an equimolar potassium ferrocyanide/potassium ferricyanide redox solution that is applied to the working electrode for determining the quantity of 17β-estradiol in the sample bound to the anti-estrogen receptor, and
a measuring device for applying voltage potentials to the working electrode and counter electrode and measuring the current flow between the working electrode and counter electrode.

11. The system of claim 10, wherein the working electrode and the counter electrode comprise metalized films.

12. The system of claim 10, wherein the working electrode and counter electrode independently comprise gold, platinum, palladium, silver, carbon, alloys thereof, and composites thereof.

13. The system of claim 11, wherein the metalized films are provided on the surface of the substrate by sputtering or coating the films on the surface and wherein the working electrode and the counter electrode are formed using laser ablation to define the dimensions of the working electrode and the counter electrode.

14. The system of claim 10, further comprising a reference electrode on the surface of the substrate, the dielectric covering a portion of the reference electrode.

15. The system of claim 10, the anti-estrogen receptor being chemically functionalized to the surface of the working electrode coated with a 3-mercaptopropionic acid (MPA) monolayer.

16. The system of claim 10, wherein the anti-estrogen receptor comprises an α-estrogen antibody.

17. The system of claim 10, wherein the sample comprises urine or tap water.

18. A detection system for detecting 17β-estradiol levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an α-estrogen antibody functionalized or chemically functionalized to a surface of the exposed portion of the working electrode, the α-estrogen antibody selectively binding to anti-17β-estradiol antibody in a sample and the 17β-estradiol once bound being detectable by measuring the current flow between the working electrode and counter electrode,
an equimolar potassium ferrocyanide/potassium ferricyanide redox solution that is applied to the working electrode for determining the quantity of 17β-estradiol in the sample bound to the α-estrogen antibody, and
a measuring device for applying voltage potentials to the working electrode and counter electrode and measuring the current flow between the working electrode and counter electrode.

19. The system of claim 18, wherein the working electrode and the counter electrode comprise metalized films, the metalized films are provided on the surface of the substrate by sputtering or coating the films on the surface and wherein the working electrode and the counter electrode are formed using laser ablation to define the dimensions of the working electrode and the counter electrode.

20. The system of claim 18, wherein the sample comprises urine or tap water.
